(19) United States
(12) Patent Application Publication
(10) Pub. No.: US 2012/0083444 A1
(43) Pub. Date: Apr. 5, 2012

(54) Emulsion Method For Preparing Low Residual Solvent Microparticles

(76) Inventors: Brenda Perkins, Birmingham, AL (US); Asima Pattanaik, Vestavia Hills, AL (US)

(21) Appl. No.: 13/247,577

(22) Filed: Sep. 28, 2011

Related U.S. Application Data
(60) Provisional application No. 61/388,049, filed on Sep. 30, 2010.

(51) Int. Cl. A61K 38/31; A61K 47/34; A61K 38/02; A61P 31/00; A61P 37/08; A61P 29/00; A61P 25/16; A61P 9/12; A61P 25/20; A61K 47/30; A61P 37/02

(52) U.S. Cl. 514/11.1; 514/772; 514/785; 514/772.3; 514/772.7; 514/1.1

(57) ABSTRACT

The method disclosed herein comprises using a non-polar alkane in the continuous phase of an emulsion process to aid in the removal of dispersed phase solvent from the microparticles. The microparticles can further be subjected to a post-production treatment process, involving a non-polar alkane suspension and a rinse, to further reduce residual dispersed phase solvent levels.

This application claims benefit of U.S. Provisional Application No. 61/388,049, filed Sep. 30, 2010, which is hereby incorporated herein by reference in its entirety.

### BACKGROUND

Microparticles are often prepared using a solvent to dissolve the polymer which forms the microparticle matrix. Typical solvents for polyesters such as lactide and/or glycolide based polymers include a variety of ICH Class I and Class II solvents, such as chlorinated solvents. Such solvents are regulated and cannot be present above certain amounts in formulations for in vivo use. In many microparticle production processes, however, residual solvent is difficult to remove. Accordingly, a need exists for methods to overcome the problem of residual solvent.

### SUMMARY

The disclosed methods and emulsions make use of a non-polar alkane in the continuous phase of the emulsion and/or an optional non-polar alkane post-production treatment to reduce the amount of residual dispersed phase solvent present in microparticles.

The emulsions disclosed herein comprise: a dispersed phase, comprising: a biocompatible polymer dispersed or dissolved in a dispersed phase solvent that comprises a C1-C4 halogenated alkane, ethyl acetate, or a combination thereof; and a continuous phase comprising a surfactant mixture and a non-polar alkane; wherein the surfactant mixture comprises at least 2% by weight of the non-polar alkane dissolved or dispersed therein; wherein the dispersed phase is dispersed in the continuous phase.

In one aspect, the disclosed methods for preparing microparticles comprise: (a) providing a first phase comprising a biocompatible polymer dispersed or dissolved in a dispersed phase solvent comprising a C1-C4 halogenated alkane, ethyl acetate, or a combination thereof; (b) providing a second phase comprising a continuous phase surfactant mixture and a non-polar alkane; wherein the surfactant mixture comprises at least 2% by weight of the non-polar alkane dissolved or dispersed therein; (c) mixing the first and second phases to form an emulsion; and (d) removing at least a portion of the dispersed phase solvent to form microparticles.

In a further aspect, the method for preparing microparticles can optionally comprise, after the formation of the microparticles: (a) combine microparticles with a surfactant mixture in a non-polar alkane to provide a dispersion; wherein the microparticles comprise at least 2% by weight residual organic solvent; (b) mixing the dispersion; (c) collecting the microparticles; (d) rinsing the microparticles; and (e) drying the microparticles.

### DETAILED DESCRIPTION

In this specification and in the claims that follow, reference will be made to a number of terms that shall be defined to have the following meanings:

Throughout this specification, unless the context requires otherwise, the word “comprise,” or variations such as “comprises” or “comprising,” will be understood to imply the inclusion of a stated integer or step or group of integers or steps but not the exclusion of any other integer or step or group of integers or steps.

The singular forms “a,” “an” and “the” include plural referents unless the context clearly dictates otherwise. Thus, for example, reference to “a bioactive agent” includes mixtures of two or more such agents, and the like.

“Biodegradable” refers to materials that erode to soluble species or that degrade under physiologic conditions to smaller units or chemical species that are, themselves, non-toxic (biocompatible) to the subject and capable of being metabolized, eliminated, or excreted by the subject.

A “bioactive agent” refers to an agent that has biological activity. The biological agent can be used to treat, diagnose, cure, mitigate, prevent (i.e., prophylactically), ameliorate, modulate, or have an otherwise favorable effect on a disease, disorder, infection, and the like. Bioactive agents also include those substances which affect the structure or function of a subject, or a pro-drug, which becomes bioactive or more bioactive after it has been placed in a predetermined physiological environment.

The term “microparticle” is used herein to refer generally to a variety of structures having sizes from about 10 nm to 2000 microns (2 millimeters) and includes microcapsule, microsphere, nanoparticle, nanocapsule, nanosphere as well as particles, in general, that are less than about 2000 microns (2 millimeters).

In one aspect, he emulsions disclosed herein can be prepared by a process that utilizes a non-polar alkane in the continuous phase. The non-polar alkane allows for removal of at least some of the dispersed phase solvent used in the dispersed phase. After the emulsion process, the microparticles can be optionally subjected to a further residual solvent removal process to further decrease the amount of residual dispersed phase solvent present in the microparticles.

The emulsions comprise: a dispersed phase, comprising: a biocompatible polymer dispersed or dissolved in a dispersed phase solvent that comprises a C1-C4 halogenated alkane, ethyl acetate, or a combination thereof; and a continuous phase comprising a surfactant mixture and a non-polar alkane; wherein the surfactant mixture comprises at least 2% by weight of the non-polar alkane dissolved or dispersed therein; wherein the dispersed phase is dispersed in the continuous phase.

Preparing the emulsion, and preparing microparticles from the emulsion, comprises: (a) providing a first phase comprising a biocompatible polymer dispersed or dissolved in a dispersed phase solvent comprising a C1-C4 halogenated alkane, ethyl acetate, or a combination thereof; (b) providing a second phase comprising a continuous phase surfactant mixture and a non-polar alkane; wherein the surfactant mixture comprises at least 2% by weight of the non-polar alkane dissolved or dispersed therein; (c) mixing the first and second phases to form an emulsion; and (d) removing at least a portion of the dispersed phase solvent to form microparticles.

The emulsions can be either single emulsions or double emulsions. A bioactive agent can be present in the dispersed phase, either dissolved or dispersed in the dispersed phase solvent, dispersed as a solid in the dispersed phase, or dissolved or dispersed in an inner aqueous, or a combination thereof. In the latter instance wherein the bioactive agent is dissolved or dispersed in an inner aqueous phase, the emulsion is a double-emulsion.

The bioactive agent can be present in the dispersed phase or in the inner aqueous phase in any suitable quantity. For example, the bioactive agent can be present in the dispersed phase or inner aqueous phase in about 1% to about 90% by weight, including without limitation, about 5%, 10%, 15%, 20%, 30%, 40%, 50%, 60%, 70%, or 80% by weight. In one aspect, the dispersed phase comprises at least 10% by weight of the biocompatible polymer.

The dispersed phase comprises a dispersed phase solvent for dissolving or dispersing the biocompatible polymer and/or the bioactive agent. The dispersed phase solvent comprises a C1-C4 halogenated alkane, ethyl acetate, or a combination thereof. The C1-C4 halogenated alkane can be any suitable solvent, including without limitation methylene chloride, chloroform, carbon tetrachloride, ethylene dichloride, ethylene chloride, 2,2,2-trichloroethane, or a mixture thereof.

The dispersed phase and/or the continuous phase can comprise one or more other solvents or components, for example, ethanol, methanol, DMSO, DMF, isopropyl alcohol, among many other solvents. Either phase can also contain other excipients, such as buffers, salts, sugars, surfactants and/or viscosity-modifying agents, or combinations thereof.

The first phase that becomes the dispersed phase in the continuous phase can be prepared by mixing, dissolving, or dispersing the polymer and/or bioactive agent in the dispersed phase solvent. The polymer can be present in the first phase in any desired weight %. For example, the polymer can be present in the second phase in about 1% to about 90% by weight, including without limitation, about 5%, 10%, 15%, 20%, 30%, 40%, 50%, 60%, 70%, or 80% by weight. In one aspect, the polymer is present in the dispersed phase in an amount of at least 10% by weight.

The first and second phases are mixed to form the emulsion. The emulsion comprises the first phase comprising the polymer (and/or bioactive agent) as the internal phase, which is substantially surrounded by the continuous phase, comprising the surfactant mixture and the non-polar alkane. The mixing of the first and second phase can be accomplished by conventional mixing, for example, by using an emulsifier or a homogenizer.

For forming a double-emulsion, a water-in-oil emulsion comprising the inner aqueous phase (comprising the bioactive agent) dispersed in the dispersed phase (comprising the dispersed phase solvent and the polymer), i.e., the primary emulsion can be mixed with the second phase comprising the surfactant mixture and the non-polar alkane. The inner aqueous phase can comprise any desirable aqueous solvent. One non-limiting example of an aqueous solvent is water. In one aspect, water can be mixed with another miscible solvent, for example, ethanol, methanol, DMSO, DMF, isopropyl alcohol, among many other water-miscible polar solvents.

Once the emulsion is formed, microparticles can be prepared from the emulsion. The microparticles are typically formed by removing at least a portion of the dispersed phase solvent . The solvent can be removed by any suitable method. In one aspect, the solvent can be removed by extracting the solvent with an extraction liquid, such as water. In other aspects, the solvent can be removed by drying, such as by spray drying, drying under reduced pressure, solvent evaporation, lyophilization, or a combination thereof.

Emulsion methods for preparing microparticles are further discussed in Jeffery, et al., “The preparation and characterization of poly(lactide-co-glycolide) microparticles. I: Oil-In-water emulsion solvent evaporation,” Int. J. Pharm. 77(2-3):169-175 (1991); Jeffery, et al., “The Preparation and Characterization of Poly(lactide-co-glycolide) Microparticles. II. The Entrapment of a Model Protein using a (Water-in-Oil)-in-Water Emulsion Solvent Evaporation Technique,” Pharm. Res. 10(3):362-368 (1993). Solvent evaporation methods are discussed Wichert, B. and Rohdewald, P. (1993) J. Microencapsulation. 10:195. Solvent extraction methods are described in U.S. Pat. No. 5,407,609, the entirety of which is incorporated herein by reference.

As discussed above, the continuous phase comprises a non-polar alkane dissolved or dispersed in a surfactant mixture. The non-polar alkane aids in the removal of the dispersed phase solvent. The non-polar alkane can also have a plasticizing effect on the microparticles particularly when the microparticles comprise lactide and/or glycolide. The non-polar alkane can also be miscible with the dispersed phase solvent, which aids in the removal of the dispersed phase solvent from the microparticles.

The non-polar alkane can be a variety of alkanes having from 1 to 24 carbons. The alkanes can be branched or unbranched, cyclic, or non-cyclic. Examples include, without limitation, pentane, cyclopentane, hexanes, cyclohexane, and heptane. “Hexanes” refers to commercially available hexanes, which includes a variety of isomers of hexane (all having the formula, C₆H₁₄), and is thus referred to as “hexanes,” rather than “hexane.”

The surfactant mixture can serve as the continuous phase and comprises at least 2% by weight of the non-polar alkane, for example, from 2% to 30%, 2% to 20%, 2% to 10%, or 2% to 5%.

A variety of surfactants can be used in the surfactant mixture. Examples of surfactants include sorbitol monostearate (also known as SPAN), sorbitan monostearate (also known as SPAN 60), sorbitan monooleate (SPAN 80), polyoxyethylene sorbitan monooleate (TWEEN 80), all of which are commercially available. It is understood and herein contemplated that the surfactant mixture can comprise any one surfactant or combination of two, three, four or more surfactants. For example, the surfactant mixture can comprise sorbitol monostearate and sorbitan monostearate, sorbitol monostearate and sorbitan monooleate; sorbitol monostearate and polyoxyethylene sorbitan monooleate, sorbitan monostearate and sorbitan monooleate, sorbitan monostearate and polyoxyethylene sorbitan monooleate, sorbitan monooleate and polyoxyethylene sorbitan monooleate, sorbitol monostearate, sorbitan monostearate, and sorbitan monooleate, sorbitol monostearate, sorbitan monostearate, and polyoxyethylene sorbitan monooleate, or any other combination of the above identified surfactants.

As briefly discussed above, the method of preparing the microparticles can further comprise (after forming the microparticles and removing at least a portion of the dispersed phase solvent) first adding microparticles to a surfactant mixture in a non-polar alkane to provide a dispersion of microparticles in the non-polar alkane solution. The surfactant can be added to the non-polar alkane prior to the addition of the microparticles. The surfactant functions to disperse the microparticles such that the non-polar alkane can effectively soak and/or penetrate the microparticle matrix. The surfactant and the non-polar alkane can be any of those discussed above in connection with the emulsion process. The non-polar alkane solution can comprise at least 0.1% surfactant, for example from 0.1% to 10%, 0.1% to 8%, 0.1% to 6%, 0.1% to 5%, or 0.1% to 2%. The non-polar alkane solution can, in other aspects, comprise at least 0.5% surfactant, for example from 0.5% to 10%, 0.5% to 8%, 0.5% to 6%, 0.5% to 5%, or 0.5% to 2%.

Prior to adding the microparticles to the non-polar alkane solution for the post-production treatment, the microparticles comprise some amount of residual dispersed phase solvent left over from the emulsion process. The additional steps for removing residual dispersed phase solvent can be useful for microparticles that comprise at least 2% by weight residual dispersed phase solvent, for example from 2% to 5%. The residual dispersed phase solvent is the solvent used as the solvent for the polymer during the microparticle production process. The non-polar alkane, in contrast, is not a solvent for the polymer from which the microparticles were formed. The non-polar alkane is also not a solvent for any bioactive agent or excipient present in the microparticles.

After adding microparticles to the non-polar alkane solution during the post-production treatment, the dispersion of microparticles in the non-polar alkane solution can be stirred for a period of time generally ranging from a 5 minutes to 4 hours, for example, from 30 minutes to 2 hours. After stirring the dispersion, the microparticles can be collected, for example by filtration or by sieve separation. Once the microparticles are collected, the microparticles can be rinsed with surfactant-free non-polar alkane, such as heptane, water, or a combination thereof, and dried. The drying step can be carried out using methods known in the art, such as spray-drying, air-drying, vacuum filtration, and the like.

In contrast to existing phase separation techniques, the disclosed methods do not involve the use of oils such as silicon oils. Silicone oils are often used in microparticle phase separation processes. However, silicone oil can be difficult to entirely remove, contaminates surfaces, and can be difficult to discard. The disclosed method also allows for the ability to exchange an ICH Class II solvent, such as dichloromethane or ethyl acetate, with a class III solvent, such as heptane. Residual non-polar alkane (such as heptane) that may be present in the microparticles after carrying out the method is not as great of a concern as residual dichloromethane or ethyl acetate.

A variety of biocompatible polymers can be used in the emulsions and methods disclosed herein. In one aspect, the biocompatible polymer can also be a biodegradable polymer. In another aspect, the biocompatible polymer can also be a biodegradable polymer. For example, the biocompatible polymer can be one or more of polyesters, polyhydroxyalkanoates, polyhydroxybutyrates, polydioxanones, polyhydroxyvalerates, polyanhydrides, polyorthoesters, polyphosphazenes, polyphosphates, polyphosphoesters, polydioxanones, polyphosphoesters, polyphosphates, polyphosphonates, polyphosphates, polyhydroxyalkanoates, polycarbonates, polyalkylcarbonates, polyorthocarbonates, polyesteramides, polyamides, polyamines, polypeptides, polyurethanes, polyalkylene alkylates, polyalkylene oxalates, polyalkylene succinates, polyhydroxy fatty acids, polyacetals, polycyanoacrylates, polyketals, polyetheresters, polyethers, polyalkylene glycols, polyalkylene oxides, polyethylene glycols, polyethylene oxides, polypeptides, polysaccharides, or polyvinyl pyrrolidones. Other non-biodegradable but durable and bioacompatible polymers include without limitation ethylene-vinyl acetate co-polymer, polytetrafluoroethylene, polypropylene, polyethylene, and the like. Likewise, other suitable non-biodegradable polymers include without limitation silicones and polyurethanes.

The biocompatible and/or biodegradable polymer can be a poly(lactide), a poly(glycolide), a poly(lactide-co-glycolide), a poly(caprolactone), a poly(orthoester), a poly(phosphazene), a poly(hydroxybutyrate) or a copolymer containing a poly(hydroxybutarate), a poly(lactide-co-caprolactone), a polycarbonate, a polyesteramide, a polyanhydride, a poly(dioxanone), a poly(alkylene alkylate), a copolymer of polyethylene glycol and a polyorthoester, a biodegradable polyurethane, a poly(amino acid), a polyamide, a polyesteramide, a polyetherester, a polyacetal, a polycyanoacrylate, a poly(oxyethylene)/poly(oxypropylene) copolymer, polyacetals, polyketals, polyphosphoesters, polyhydroxyvalerates or a copolymer containing a polyhydroxyvalerate, polyalkylene oxalates, polyalkylene succinates, poly(maleic acid), and copolymers, terpolymers, combinations, or blends thereof.

The biocompatible or biodegradable polymer can comprise any lactide residue, including all racemic and stereospecific forms of lactide, including, but not limited to, L-lactide, D-lactide, and D,L-lactide, or a mixture thereof. Useful polymers comprising lactide include, but are not limited to poly(L-lactide), poly(D-lactide), and poly(DL-lactide); and poly(lactide-co-glycolide), including poly(L-lactide-co-glycolide), poly(D-lactide-co-glycolide), and poly(DL-lactide-co-glycolide); or copolymers, terpolymers, combinations, or blends thereof. Lactide/glycolide polymers can be conveniently made by melt polymerization through ring opening of lactide and glycolide monomers. Additionally, racemic DL-lactide, L-lactide, and D-lactide polymers are commercially available. The L-polymers are more crystalline and resorb slower than DL-polymers. In addition to copolymers comprising glycolide and DL-lactide or L-lactide, copolymers of L-lactide and DL-lactide are commercially available. Homopolymers of lactide or glycolide are also commercially available.

When the biodegradable and/or biocompatible polymer is poly(lactide-co-glycolide), poly(lactide), or poly(glycolide), the amount of lactide and glycolide in the polymer can vary. In a further aspect, the biodegradable polymer contains 0 to 100 mole %, 40 to 100 mole %, 50 to 100 mole %, 60 to 100 mole %, 70 to 100 mole %, or 80 to 100 mole % lactide and from 0 to 100 mole %, 0 to 60 mole %, 10 to 40 mole %, 20 to 40 mole %, or 30 to 40 mole % glycolide, wherein the amount of lactide and glycolide is 100 mole %. In a further aspect, the biodegradable polymer can be poly(lactide), 95:5 poly(lactide-co-glycolide) 85:15 poly(lactide-co-glycolide), 75:25 poly(lactide-co-glycolide), 65:35 poly(lactide-co-glycolide), or 50:50 poly(lactide-co-glycolide), where the ratios are mole ratios.

The biodegradable and/or biocompatible polymer can also be a poly(caprolactone) or a poly(lactide-co-caprolactone). The polymer can be a poly(lactide-caprolactone), which, in various aspects, can be 95:5 poly(lactide-co-caprolactone), 85:15 poly(lactide-co-caprolactone), 75:25 poly(lactide-co-caprolactone), 65:35 poly(lactide-co- caprolactone), or 50:50 poly(lactide-co-caprolactone), where the ratios are mole ratios.

A variety of bioactive agents or other excipients can be used. Examples include without limitation small molecules, peptides, oligopeptides (e.g., octreotide), proteins such as hormones, enzymes, antibodies, receptor binding proteins, antibody fragments, antibody conjugates, nucleic acids such as aptamers, iRNA, siRNA, microRNA, DNA , RNA, antisense nucleic acid or the like, antisense nucleic acid analogs or the like, VEGF inhibitors, macrocyclic lactones,dopamine agonists, dopamine antagonists, low-molecular weight compounds, high-molecular-weight compounds, or conjugated bioactive agents.

Other bioactive agents can include anabolic agents, antacids, anti-asthmatic agents, anti-cholesterolemic and anti-lipid agents, anti-coagulants, anti-convulsants, anti-diarrheals, anti-emetics, anti-infective agents including antibacterial and antimicrobial agents, anti-inflammatory agents, anti-manic agents, antimetabolite agents, anti-nauseants, anti-neoplastic agents, anti-obesity agents, antipsychotics, anti-pyretic and analgesic agents, anti-spasmodic agents, anti-thrombotic agents, anti-tussive agents, anti-uricemic agents, anti-anginal agents, antihistamines, appetite suppressants, biologicals, cerebral dilators, coronary dilators, bronchiodilators, cytotoxic agents, decongestants, diuretics, diagnostic agents, erythropoietic agents, expectorants, gastrointestinal sedatives, hyperglycemic agents, hypnotics, hypoglycemic agents, immunomodulating agents, ion exchange resins, laxatives, mineral supplements, mucolytic agents, neuromuscular drugs, peripheral vasodilators, psychotropics, sedatives, stimulants, thyroid and anti-thyroid agents, tissue growth agents, uterine relaxants, vitamins, or antigenic materials.

Still other bioactive agents include androgen inhibitors, polysaccharides, growth factors, hormones, anti-angiogenesis factors, dextromethorphan, dextromethorphan hydrobromide, noscapine, carbetapentane citrate, chlophedianol hydrochloride, chlorpheniramine maleate, phenindamine tartrate, pyrilamine maleate, doxylamine succinate, phenyltoloxamine citrate, phenylephrine hydrochloride, phenylpropanolamine hydrochloride, pseudoephedrine hydrochloride, ephedrine, codeine phosphate, codeine sulfate morphine, mineral supplements, cholestryramine, N-acetylprocainamide, acetaminophen, aspirin, ibuprofen, phenyl propanolamine hydrochloride, caffeine, guaifenesin, aluminum hydroxide, magnesium hydroxide, peptides, polypeptides, proteins, amino acids, hormones, interferons, cytokines, and vaccines.

Representative drugs that can be used as bioactive agents include, but are not limited to, peptide drugs, protein drugs, therapeutic antibodies, anticalins, desensitizing materials, antigens, anti-infective agents such as antibiotics, antimicrobial agents, antiviral, antibacterial, antiparasitic, antifungal substances and combination thereof, antiallergenics, androgenic steroids, decongestants, hypnotics, steroidal anti-inflammatory agents, anti-cholinergics, sympathomimetics, sedatives, miotics, psychic energizers, tranquilizers, vaccines, estrogens, progestational agents, humoral agents, prostaglandins, analgesics, antispasmodics, antimalarials, antihistamines, cardioactive agents, anti-inflammatory agents, nonsteroidal anti-inflammatory agents, antiparkinsonian agents, antihypertensive agents, β-adrenergic blocking agents, nutritional agents, anti-TNF agents and the benzophenanthridine alkaloids. The agent can further be a substance capable of acting as a stimulant, sedative, hypnotic, analgesic, anticonvulsant, and the like.

Other bioactive agents include but are not limited to analgesics such as acetaminophen, acetylsalicylic acid, and the like; anesthetics such as lidocaine, xylocaine, and the like; anorexics such as dexadrine, phendimetrazine tartrate, and the like; antiarthritics such as methylprednisolone, ibuprofen, and the like; antiasthmatics such as terbutaline sulfate, theophylline, ephedrine, and the like; antibiotics such as sulfisoxazole, penicillin G, ampicillin, cephalosporins, amikacin, gentamicin, tetracyclines, chloramphenicol, erythromycin, clindamycin, isoniazid, rifampin, and the like; antifungals such as amphotericin B, nystatin, ketoconazole, and the like; antivirals such as acyclovir, amantadine, and the like; anticancer agents such as cyclophosphamide, methotrexate, etretinate, and the like; anticoagulants such as heparin, warfarin, and the like; anticonvulsants such as phenytoin sodium, diazepam, and the like; antidepressants such as isocarboxazid, amoxapine, and the like; antihistamines such as diphenhydramine HCl, chlorpheniramine maleate, and the like; antipsychotics such as clozapine, haloperidol, carbamazepine, gabapentin, topimarate, bupropion, sertraline, alprazolam, buspirone, risperidone, aripiprazole, olanzapine, quetiapine, ziprasidone, iloperidone, and the like; hormones such as insulin, progestins, estrogens, corticoids, glucocorticoids, androgens, and the like; tranquilizers such as thorazine, diazepam, chlorpromazine HCl, reserpine, chlordiazepoxide HCl, and the like; antispasmodics such as belladonna alkaloids, dicyclomine hydrochloride, and the like; vitamins and minerals such as essential amino acids, calcium, iron, potassium, zinc, vitamin B12, and the like; cardiovascular agents such as prazosin HCl, nitroglycerin, propranolol HCl, hydralazine HCl, pancrelipase, succinic acid dehydrogenase, and the like; peptides and proteins such as LHRH, somatostatin, calcitonin, growth hormone, glucagon-like peptides, growth releasing factor, angiotensin, FSH, EGF, bone morphogenic protein (BMP), erythopoeitin (EPO), interferon, interleukin, collagen, fibrinogen, insulin, Factor VIII, Factor IX, Enbrel®, Rituxan®, Herceptin®, alpha-glucosidase, Cerazyme/Ceredose®, vasopressin, ACTH, human serum albumin, gamma globulin, structural proteins, blood product proteins, complex proteins, enzymes, antibodies, monoclonal antibodies, and the like; prostaglandins; nucleic acids; carbohydrates; fats; narcotics such as morphine, codeine, and the like, psychotherapeutics; anti-malarials, L-dopa, diuretics such as furosemide, spironolactone, and the like; antiulcer drugs such as rantidine HCl, cimetidine HCl, and the like.

The bioactive agent can also be an immunomodulator, including, for example, cytokines, interleukins, interferon, colony stimulating factor, tumor necrosis factor, and the like; allergens such as cat dander, birch pollen, house dust mite, grass pollen, and the like; antigens of bacterial organisms such as Streptococcus pneumoniae, Haemophilus influenzae, Staphylococcus aureus, Streptococcus pyrogenes, Corynebacterium diphteriae, Listeria monocytogenes, Bacillus anthracis, Clostridium tetani, Clostridium botulinum, Clostridium perfringens. Neisseria meningitides, Neisseria gonorrhoeae, Streptococcus mutans. Pseudomonas aeruginosa, Salmonella typhi, Haemophilus parainfluenzae, Bordetella pertussis, Francisella tularensis, Yersinia pestis, Vibrio cholerae, Legionella pneumophila, Mycobacterium tuberculosis, Mycobacterium leprae, Treponema pallidum, Leptspirosis interrogans, Borrelia burgddorferi, Campylobacter jejuni, and the like; antigens of such viruses as smallpox, influenza A and B, respiratory synctial, parainfluenza, measles, HIV, SARS, varicella-zoster, herpes simplex 1 and 2, cytomeglavirus, Epstein-Barr, rotavirus, rhinovirus, adenovirus, papillomavirus, poliovirus, mumps, rabies, rubella, coxsackie viruses, equine encephalitis, Japanese encephalitis, yellow fever, Rift Valley fever, lymphocytic choriomeningitis, hepatitis B, and the like; antigens of such fungal, protozoan, and parasitic organisms such as Cryptococcuc neoformans, Histoplasma capsulatum, Candida albicans, Candida tropicalis, Nocardia asteroids, Rickettsia ricketsii, Rickettsia typhi, Mycoplasma pneumoniae, Chlamyda psittaci, Chlamydia trachomatis, Plasmodium falciparum, Trypanasoma brucei, Entamoeba histolytica, Toxoplasma gondii, Trichomonas vaginalis, Schistosoma mansoni, and the like. These antigens can be in the form of whole killed organisms, peptides, proteins, glycoproteins, carbohydrates, or combinations thereof.

The bioactive agent can also comprise an antibiotic. The antibiotic can be, for example, one or more of Amikacin, Gentamicin, Kanamycin, Neomycin, Netilmicin, Streptomycin, Tobramycin, Paromomycin, Ansamycins, Geldanamycin, Herbimycin, Carbacephem, Loracarbef, Carbapenems, Ertapenem, Doripenem, Imipenem/Cilastatin, Meropenem, Cephalosporins (First generation), Cefadroxil, Cefazolin, Cefalotin or Cefalothin, Cefalexin, Cephalosporins (Second generation), Cefaclor, Cefamandole, Cefoxitin, Cefprozil, Cefuroxime, Cephalosporins (Third generation), Cefixime, Cefdinir, Cefditoren, Cefoperazone, Cefotaxime, Cefpodoxime, Ceftazidime, Ceftibuten, Ceftizoxime, Ceftriaxone, Cephalosporins (Fourth generation), Cefepime, Cephalosporins (Fifth generation), Ceftobiprole, Glycopeptides, Teicoplanin, Vancomycin, Macrolides, Azithromycin, Clarithromycin, Dirithromycin, Erythromycin, Roxithromycin, Troleandomycin, Telithromycin, Spectinomycin, Monobactams, Aztreonam, Penicillins, Amoxicillin, Ampicillin, Azlocillin, Carbenicillin, Cloxacillin, Dicloxacillin, Flucloxacillin, Mezlocillin, Meticillin, Nafcillin, Oxacillin, Penicillin, Piperacillin, Ticarcillin, Polypeptides, Bacitracin, Colistin, Polymyxin B, Quinolones, Ciprofloxacin, Enoxacin, Gatifloxacin, Levofloxacin, Lomefloxacin, Moxifloxacin, Norfloxacin, Ofloxacin, Trovafloxacin, Sulfonamides, Mafenide, Prontosil (archaic), Sulfacetamide, Sulfamethizole, Sulfanilimide (archaic), Sulfasalazine, Sulfisoxazole, Trimethoprim, Trimethoprim-Sulfamethoxazole (Co-trimoxazole) (TMP-SMX), Tetracyclines, including Demeclocycline, Doxycycline, Minocycline, Oxytetracycline, Tetracycline, and others; Arsphenamine, Chloramphenicol, Clindamycin, Lincomycin, Ethambutol, Fosfomycin, Fusidic acid, Furazolidone, Isoniazid, Linezolid, Metronidazole, Mupirocin, Nitrofurantoin, Platensimycin, Pyrazinamide, Quinupristin/Dalfopristin, Rifampicin (Rifampin in U.S.), Tinidazole, Ropinerole, Ivermectin, Moxidectin, Afamelanotide, Cilengitide, or a combination thereof. In one aspect, the bioactive agent can be a combination of Rifampicin (Rifampin in U.S.) and Minocycline.

Various modifications and variations can be made to the methods and emulsions described herein. Other aspects of the methods and emulsions described herein will be apparent from consideration of the specification and practice of the methods and emulsions disclosed herein. It is intended that the specification and examples be considered as exemplary.

What is claimed is:

1. An emulsion, comprising:
a dispersed phase, comprising: a biocompatible polymer dispersed or dissolved in a dispersed phase solvent comprising a C₁-C₄ halogenated alkane, ethyl acetate, or a combination thereof; and
a continuous phase, comprising: a surfactant mixture and a non-polar alkane; wherein the surfactant mixture comprises at least 2% by weight of the non-polar alkane dissolved or dispersed therein;
wherein the dispersed phase is dispersed in the continuous phase.

2. The emulsion of claim 1, wherein the surfactant mixture comprises sorbitan monostearate, sorbitan monooleate, polyoxyethylene sorbitan monooleate, or a combination thereof.

3. The emulsion of claim 1, wherein the biocompatible polymer comprises poly(lactide), poly(glycolide), poly(lactide-co-glycolide), poly(caprolactone), poly(lactide-co-caprolactone), polyethylene glycol, or a copolymer, blend, or mixture thereof.

4. The emulsion of claim 1, wherein the dispersed phase further comprises a bioactive agent.

5. The emulsion of claim 4, wherein the bioactive agent is water-soluble.

6. The emulsion of claim 4, wherein the bioactive agent is an oligopeptide.

7. The emulsion of any of claims 4, wherein the bioactive agent is octreotide.

8. The emulsion of claim 1, wherein the dispersed phase comprises at least 10% by weight of the biocompatible polymer.

9. The emulsion of claim 1, wherein the dispersed phase solvent comprises methylene chloride, chloroform, carbon tetrachloride, ethylene dichloride, ethylene chloride, 2,2,2-trichloroethane, or a mixture thereof.

10. The emulsion of claim 1, wherein the dispersed phase solvent comprises ethyl acetate.

11. The emulsion of claim 1, wherein the non-polar alkane comprises pentane, cyclopentane, hexanes, cyclohexane, heptane, or a combination thereof.

12. The emulsion of claim 1, wherein the non-polar alkane comprises heptane.

13. A method for preparing microparticles, comprising:
(a) providing a first phase comprising a biocompatible polymer dispersed or dissolved in a dispersed phase solvent comprising a C₁-C₄ halogenated alkane, ethyl acetate, or a combination thereof;
(b) providing a second phase comprising a continuous phase surfactant mixture and a non-polar alkane; wherein the surfactant mixture comprises at least 2% by weight of the non-polar alkane dissolved or dispersed therein;
(c) mixing the first and second phases to form an emulsion; and
(d) removing at least a portion of the dispersed phase solvent to form microparticles.

14. The method of claim 13 wherein the first phase further comprises a bioactive agent.

15. The method of claim 13 wherein the first phase further comprises, dispersed therein, an inner aqueous phase comprising a bioactive agent dissolved or dispersed therein.

16. The method of claim 13, wherein the first phase further comprises, dispersed therein, a solid bioactive agent.

17. The method of claim 13, wherein the bioactive agent is an oligopeptide.

18. The method of claim 13, wherein the bioactive agent is octreotide.

19. The method of claim 13, wherein the first phase comprises at least 10% by weight of the biocompatible polymer.

20. The method of claim 13, wherein the surfactant mixture comprises sorbitan monostearate, sorbitan monooleate, polyoxyethylene sorbitan monooleate, or a combination thereof.

21. The method of claim 13, wherein the biocompatible polymer comprises poly(lactide), poly(glycolide), poly(lactide-co-glycolide), or a copolymer, blend, or mixture thereof.

22. The method of claim 13, wherein the dispersed phase solvent comprises methylene chloride, chloroform, carbon tetrachloride, ethylene dichloride, ethylene chloride, 2,2,2-trichloroethane, or a mixture thereof.

23. The method of claim 13, wherein the dispersed phase solvent comprises ethyl acetate.

24. The method of claim 13, wherein the non-polar alkane comprises pentane, cyclopentane, hexanes, cyclohexane, heptane, or a combination thereof.

25. The method of claim 13, wherein the non-polar alkane comprises heptane.
